(19) United States
(12) Patent Application Publication
(10) Pub. No.: US 2017/0021126 A1
(43) Pub. Date: Jan. 26, 2017

(54) REDUCING VENTILATOR-INDUCED LUNG INJURY

(71) Applicant: The Trustees of the Stevens Institute of Technology, Hoboken, NJ (US)

(72) Inventor: Carrie E. Perlman, New York, NY (US)

(21) Appl. No.: 15/288,495

(22) Filed: Oct. 7, 2016

Related U.S. Application Data
(63) Continuation-in-part of application No. 13/650,759, filed on Oct. 12, 2012, now Pat. No. 9,504,796.
(60) Provisional application No. 61/547,133, filed on Oct. 14, 2011.

(51) Int. Cl. A61M 16/20; A61M 16/08; A61M 16/00

(52) U.S. Cl. CPC A61M 16/203; A61M 16/0009; A61M 16/0875; A61M 16/0006; A61M 2016/0027

(57) ABSTRACT

Methods are provided for protecting against ventilation-induced lung injury by promoting equitable liquid distribution in a lung with alveolar flooding, in which flooded and aerated alveoli are interspersed. Since ventilation injuriously over-expands aerated alveoli adjacent to flooded alveoli and a pressure barrier is responsible for trapping liquid in discrete alveoli, the present invention provides various means for overcoming the pressure barrier to, in turn, promote equitable redistribution of flooding liquid amongst alveoli, reduce the number of aerated alveoli located adjacent to flooded alveoli and reduce ventilation injury of the lung. These means of overcoming the pressure barrier include: (i) use of accelerated deflation during mechanical ventilation; and ii) high frequency (>50 Hz) vibration of the lung.

### CROSS-REFERENCE TO RELATED APPLICATIONS

The present application is a continuation-in-part of U.S. patent application Ser. No. 13/650,759, filed Oct. 12, 2012, which claims the benefit of U.S. Provisional Patent Application No. 61/547,133, filed on Oct. 14, 2011, both of which are incorporated by reference herein in their entireties.

### FIELD OF THE INVENTION

The present invention relates to methods for promoting equitable liquid distribution amongst pulmonary alveoli in the presence of alveolar flooding, so as to reduce ventilator-induced lung injury.

### BACKGROUND OF THE INVENTION

Physiology and Pathophysiology

Lung Physiology.

The terminal airspaces of the lungs, the alveoli, are lined with a thin liquid layer. Thus there is an air-liquid interface in the lungs that has an associated surface tension. To reduce the surface tension, alveolar type II epithelial cells release surfactant—an aggregate of phospholipids and proteins—into the liquid lining layer. The surfactant adsorbs to and reduces surface tension at the air-liquid interface. By lowering surface tension, surfactant reduces the pressure required to keep the lungs inflated and reduces the work of breathing.

ARDS.

Acute respiratory distress syndrome (ARDS) can result from a variety of initial insults. Regardless of initial insult, inflammation is present in the lungs. With inflammation, permeability of the alveolar-capillary membrane increases and liquid leaks out of the vasculature. When enough liquid escapes from the vessels, liquid begins to enter the alveoli, a condition known as alveolar edema. Initially, discrete alveoli in the dependent (bottom portion of the) lung become flooded and are interspersed with alveoli that remain aerated. With disease progression, most alveoli in the dependent lung become flooded; in the nondependent lung, some alveoli become flooded and are interspersed with other alveoli that remain aerated.

With alveolar edema, the additional liquid in the airspace effectively thickens the alveolar-capillary membrane across which oxygen and carbon dioxide are exchanged, and therefore impairs gas exchange. Also in ARDS with alveolar edema, lung compliance is reduced, likely due to both airspace flooding and increased surface tension, and the reduced compliance further impairs gas exchange.

ARDS patients are treated by mechanical ventilation, which assists gas exchange and keeps patients alive but often causes an over-distension injury (ventilator-induced lung injury, VILI) that exacerbates the underlying lung disease and prevents patient recovery. It is now standard protocol to deliver a low tidal (breath) volume, V_{T}, that has been shown to decrease mortality. However, mortality still exceeds 35%.

It has been hoped that administration of exogenous surfactant would reduce surface tension, increase lung compliance, improve gas exchange and reduce VILI. Thus, multiple randomized clinical trials have tested tracheal administration of exogenous surfactant in ARDS patients. However, exogenous surfactant administration has not altered clinical outcome.

In VILI, the site of over-distension injury is likely in aerated alveoli adjacent to flooded alveoli. In flooded alveoli, the air-liquid interface forms a concave meniscus. Due to pressure drop across the meniscus, flooded alveoli are shrunken and, due to interdependence, adjacent aerated alveoli are expanded. Lung inflation during mechanical ventilation exacerbates the over-expansion of aerated alveoli adjacent to flooded alveoli. Further, the flooded and aerated alveoli exhibit normal and reduced compliance, respectively. This difference in expansion mechanics between adjacent flooded and aerated alveoli may contribute to the ventilation induced over-expansion of aerated alveoli located adjacent to flooded alveoli.

Neonatal Respiratory Distress Syndrome (RDS).

The fetal lung is entirely filled with fluid. When babies are born prematurely, significant fluid remains in the lung such that aeration is heterogeneous. In this condition of neonatal RDS, as in ARDS, mechanical ventilation is often used to assist gas exchange and mechanical ventilation causes injury—likely to aerated areas adjacent to flooded areas. Also as in ARDS, ventilation injury is proportional to surface tension.

Surfactant production increases markedly during the third trimester of gestation. Premature babies born prior to or early during the third trimester used not to survive. Since the 1980's, tracheal instillation of exogenous surfactant has enabled such premature babies to live and reduced the injury caused by mechanical ventilation. However, mechanical ventilation is still injurious and there remains room for improvement in the clinical treatment of neonatal RDS.

High Frequency Modes of Lung Treatment.

For various objectives such as loosening/clearing airway mucus and improved mechanical ventilation, the lung has sometimes been subjected to percussion and to high frequency ventilation. Devices designed to implement such treatments, and the frequencies at which they operate, include: pneumatically and electrically powered percussors; intrapulmonary percussive ventilation (1.7-5 Hz); flutter valve therapy; high-frequency chest wall oscillation (5-25 Hz); high frequency positive-pressure ventilation (1-1.8 Hz); high-frequency jet ventilation (≦10 Hz); high-frequency oscillatory ventilation (HFOV, 1-50 Hz); high-frequency flow interruption (≦15 Hz, where the flow interruption occurs during inspiration, not expiration); and high-frequency percussive ventilation (≦2 Hz). None of these ‘high-frequency’ treatments operate at a frequency greater than 50 Hz.

Active Deflation.

Certain existing modes of ventilation have incorporated active deflation. Although now out of use, ventilation with negative end-expiratory pressure (NEEP)—available on Puritan Bennett AP series and Bird Mark 7 and 8 ventilators—can be achieved by using a Venturi tube to actively draw air out of the airways and lower the minimal tracheal pressure at end-expiration below atmospheric pressure. In a Venturi tube, a high pressure gas jet is forced through a small orifice at the tube end while there is a second port in the tube for entrance of a different gas at lower velocity. The jet accelerates the lower velocity gas by entrainment.

High-frequency oscillatory ventilation uses an oscillator to move a diaphragm at one end of a chamber. On its forward stroke the oscillator moves air into the lungs; on its backward stroke it actively pulls air out of the lungs. HFOV is most frequently used in neonatal ventilation, although it is used in adults as well.

### SUMMARY OF THE INVENTION

In one aspect of the present invention, an active, accelerated deflation method is applied during mechanical ventilation of the edematous lung to promote equitable edema liquid redistribution between alveoli. An embodiment of the present invention includes an apparatus for generating ventilation pressure waveforms with such accelerated deflation.

### BRIEF DESCRIPTION OF THE DRAWINGS

For a more complete understanding of the present invention, reference is made to the following detailed description of exemplary embodiments considered in conjunction with the accompanying drawings, in which:

FIG. 1 is a schematic illustration of a novel analysis of regional liquid phase pressures in a flooded alveolus adjacent to an aerated alveolus, made according to an embodiment of the present invention;

FIG. 2A is series of microphotographs showing a control, aerated area of a lung where a liquid has been microinjected periodically into a group of surface alveoli to avoid persistence of alveolar flooding in an experiment performed to demonstrate an embodiment of the present invention;

FIG. 2B is a series of microphotographs showing a flooded, experimental area of a lung where a liquid has been microinjected continuously into a group of surface alveoli to generate a local model of alveolar edema, in an experiment performed to demonstrate an embodiment of the present invention;

FIG. 2C is a graph showing grouped fluorescence-level data indicative of ventilation-induced injury for two different sets of ventilation pressure limits in an experiment performed to demonstrate an embodiment of the present invention;

FIG. 3 is a pair of micrographs depicting a flooded alveolus that has spontaneously cleared;

FIGS. 4A and 4B are a pair of enhanced micrographs showing a local alveolar edema model and a global permeability edema model in experiments performed to demonstrate an embodiment of the present invention;

FIG. 5 is a schematic block diagram of an apparatus for the generation of custom ventilation pressure waveforms, according to an embodiment of the present invention;

FIGS. 6A and 6B are a pairing of a set of micrographs and a graph comparing clearance of alveoli in a local edema model by ventilation using a sinusoidal pressure waveform and ventilation using a sawtooth waveform, according to embodiments of the present invention;

FIGS. 6C and 6D are a pairing of a set of micrographs and a graph comparing clearance of alveoli in a global permeability edema model by ventilation using a sinusoidal pressure waveform and ventilation using a sawtooth waveform, according to embodiments of the present invention;

FIG. 7 is a pair of graphs showing the effect of vacuum acceleration during deflation on pressure ventilation waveforms generated according to embodiments of the present invention with the apparatus of FIG. 5;

FIGS. 8A and 8B are a pair of schematic images generated by a computational fluid dynamics model, representing the effect of vibrating a flooded alveolus according to an embodiment of the present invention;

FIGS. 8C, 8D, and 8E are a group of schematic drawings illustrating a conceptual model of the effect of vibration on flooded alveolar surface tension, performed according to an embodiment of the present invention;

FIGS. 9A and 9B are a pairing of an enhanced micrograph and a graph indicating that surface tension is spatially uniform;

FIGS. 10A and 10B are a pairing of a set of micrographs and a graph illustrating alveolar liquid clearance by vibration of the lung surface in a local edema model according to an embodiment of the present invention;

FIGS. 10C and 10D are a pairing of a set of micrographs and a graph illustrating alveolar liquid clearance by vibration of the lung surface in a global permeability edema model according to an embodiment of the present invention;

FIG. 11 is a graph showing the effect of vacuum pressure, P_{VAC}, on peak lung deflation rate;

FIG. 12A is a series of graphs showing the effect of vacuum acceleration of deflation on ventilation pressure trace during ventilation with three positive end-expiratory pressure (PEEP) settings and two tidal volume (V_{T}) settings;

FIG. 12B is a graph showing grouped data for the effect of vacuum acceleration of deflation on peak deflation rate during ventilation with three PEEP settings and two V_{T} settings;

FIG. 13A is a series of micrographs showing clearance of alveolar liquid from flooded alveoli in a local edema model when ventilated using accelerated deflation without and with application of vacuum pressure;

FIG. 13B is a graph showing grouped data for the change in the percent of flooded alveoli present during ventilation without and with application of vacuum pressure;

FIG. 13C is a graph showing grouped data for the percent of flooded alveoli that are cleared during ventilation without and with application of vacuum pressure;

FIG. 13D is a graph with grouped data showing the effect of vacuum acceleration of deflation on the clearance of flooded alveoli during ventilation with three PEEP settings and two V_{T} settings;

FIG. 14A is a graph showing the relationship between the percent of flooded alveoli cleared and peak deflation rate;

FIG. 14B is a graph showing the relationship between the percent of flooded alveoli cleared and maximum airway pressure applied, for a subset of cases in which peak deflation rate exceeds a threshold value;

FIG. 15A is a graph showing decreasing flooding heterogeneity with increasing ventilation cycles; and

FIG. 15B is a graph comparing flooding heterogeneity with the percent of flooded alveoli.

### DETAILED DESCRIPTION OF THE INVENTION

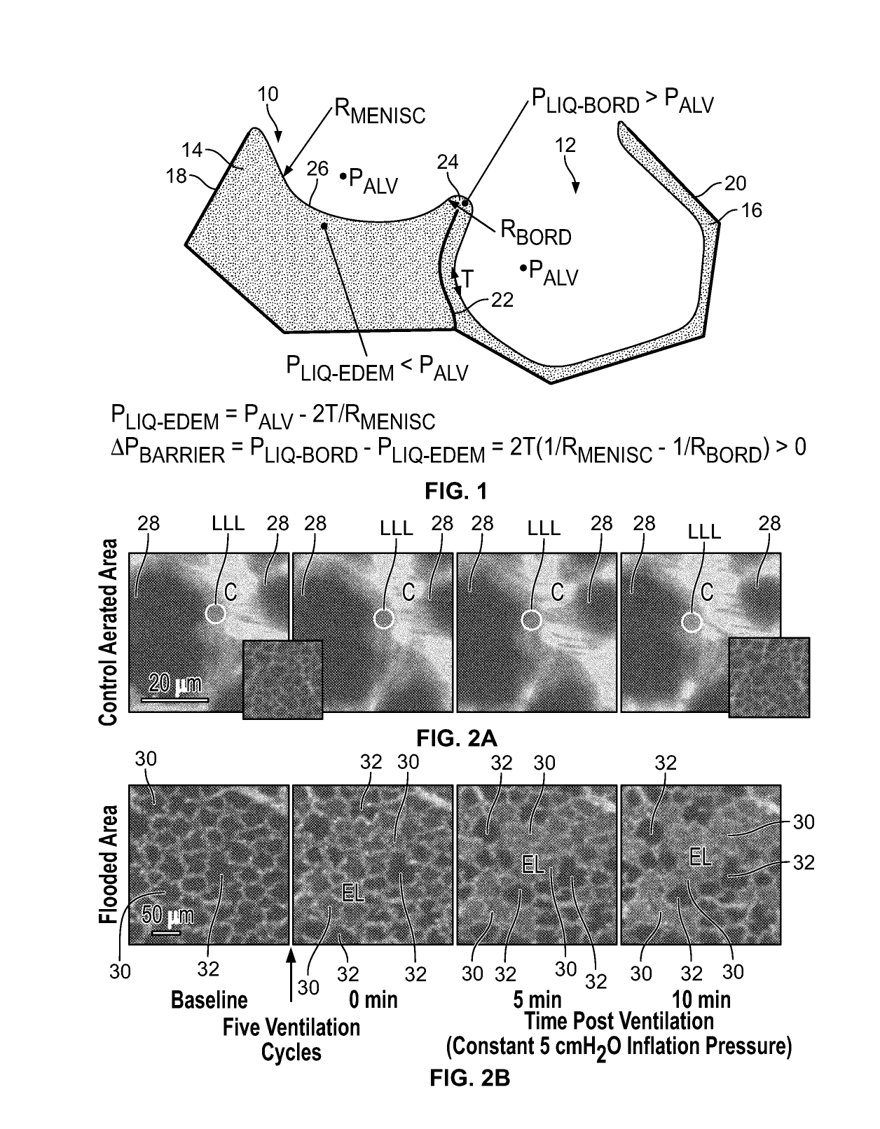

FIG. 1 is a schematic drawing of regional liquid-phase pressures in a flooded (i.e., edematous) alveolus 10 adjacent to an aerated alveolus 12, according to a novel analysis of the mechanics of alveolar flooding by the inventor of the present invention. The shaded areas 14, 16 represent liquid. The dark lines represent alveolar wall 18, 20 22, where alveolar wall 22 is also a septum 22 between the flooded alveolus 10 and the aerated alveolus 12. As the liquid phase is continuous between alveoli, such as alveoli 10, 12, the flooding liquid 14 of the flooded alveolus 10 is continuous with the liquid lining layer 16 of the aerated alveolus 12.

Considering the pressures across the meniscus of the flooded alveolus 10, and according to the Laplace relation, P_{ALV}>P_{LIQ.EDEM}, where P_{ALV} is transpulmonary pressure and P_{LIQ.EDEM} is liquid pressure in the center of the flooded alveolus, and the difference between the two pressures is proportional to interfacial surface tension, T. Thus, pressure is greater in the aerated alveolus 12, where air pressure is the same P_{ALV} as above the meniscus of the flooded alveolus 10, than in the center of the flooded alveolus 10, to a degree proportional to T. Due to this pressure imbalance, the septum 22 between the two alveoli 10, 12 bows into the flooded alveolus 10 causing that alveolus 10 to shrink and the aerated alveolus 12 to be expanded, to a degree proportional to T. Also due to the Laplace relation, P_{LIQ.BORD}>P_{ALV}, where P_{LIQ.BORD} is liquid pressure at the border 24 between the flooded and aerated alveoli 10, 12. Thus P_{LIQ.BORD}>P_{LIQ.EDEM}, forming a pressure barrier, ΔP_{BARRIER}=P_{LIQ.BORD}−P_{LIQ.EDEM}, opposing liquid flow out of the flooded alveolus 10.

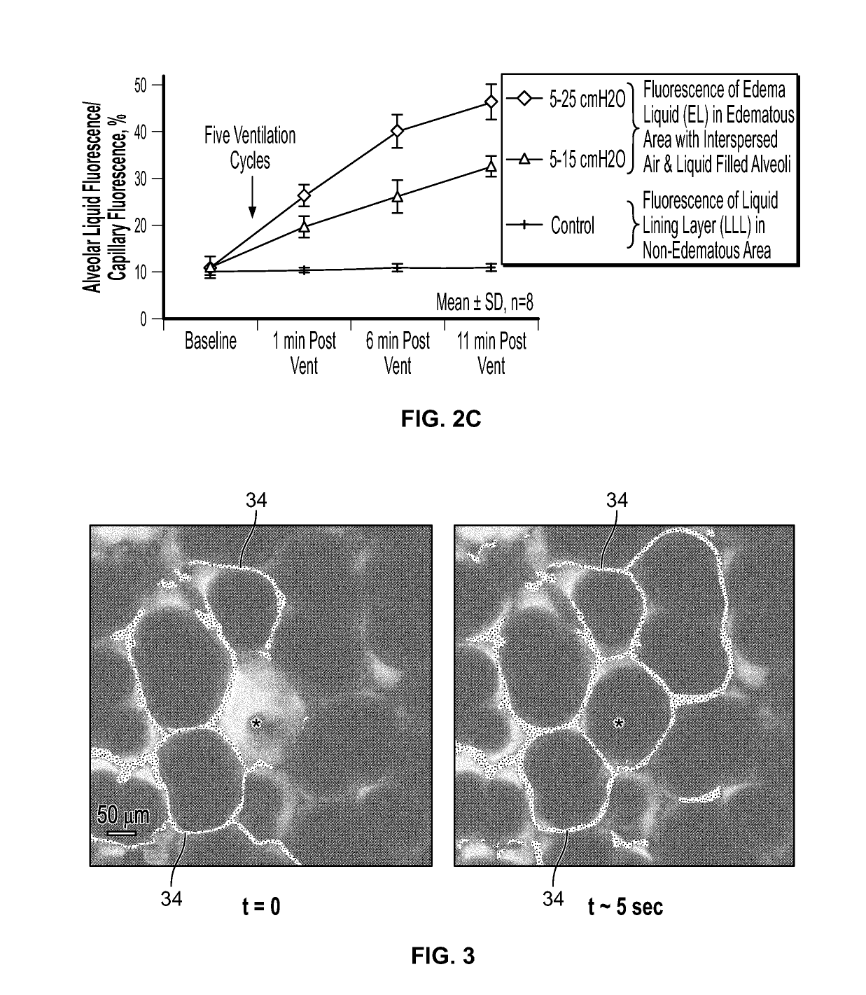

As the degree of over-expansion of the aerated alveolus is proportional to T, the degree of over-expansion is exacerbated during ventilation by lung inflation, which increases T. And the exacerbation is injurious. FIGS. 2A-2C demonstrate that in the presence of interspersed aerated and flooded alveoli, ventilation causes sustained injury (i.e., VILI) to the alveolar-capillary membrane.

The micrographs of FIG. 2B show an area of an isolated, perfused rat lung in which a local edema model, with heterogeneous alveolar flooding, was generated. Fluorescein (34 micromolar, μM) was included in the perfusate to label the capillaries (C). Non-fluorescent normal saline with 5% albumin was microinjected into a group of surface alveoli. Microinjections were delivered continuously such that some alveoli were stably flooded where as other alveoli remained aerated. The area was imaged by confocal microscopy at tracheal entrance pressure, P_{AW} (equal to P_{ALV} in the constantly inflated lung), of 5 centimeters of water (cmH₂O). The lung was ventilated five times between P_{AW} of 5 and 25 cmH₂O and then returned to a constant P_{AW} of 5 cmH₂O for 10 min of additional imaging. The five ventilation breaths generated an over-distension injury to the alveolar-capillary membrane, as evidenced by fluorescein escape from the vasculature and entrance into the alveolar liquid. Further, over the 10 min of post-ventilation imaging period with the lung held at constant, low inflation volume, alveolar liquid fluorescence increased progressively, indicating the injury was not transient, but sustained. Exemplary flooded and aerated alveoli are shown as gray and dark areas 30 and 32, respectively, in post-ventilation images. In the baseline (left-most) image of the flooded area, flooded and aerated alveoli 30 and 32 are not distinguishable as the flooding liquid was not fluorescent at baseline.

The micrographs of FIG. 2A show a control area of a rat lung in which fluorescein was included in the perfusate and alveolar microinjections were delivered periodically, such that liquid cleared from alveoli between injections and the area did not become flooded. White circles label an area of the alveolar liquid lining layer (LLL) and insets show a lower magnification of the alveolar field. Post ventilation, LLL fluorescence remained unchanged, indicating that ventilation did not injure the alveolar-capillary membrane in the control, aerated region.

FIG. 2C is a graph, showing grouped data for the tests described with relation to FIGS. 2A and 2B for two different sets of ventilation pressure limits, demonstrating that the means of detecting VILI discussed above is sensitive to the degree of injury resulting from the mechanical ventilation of the lung.

As ventilation injures aerated alveoli adjacent to flooded alveoli, more equitable redistribution of flooding liquid amongst alveoli would, by equalizing forces across more septa, reduce over-distension injury. To promote equitable flooding liquid distribution, the cause of liquid trapping in discrete alveoli must be understood.

Flooded alveoli are generally stable, but occasionally clear. When flooded alveoli clear, they do so spontaneously, unpredictably and instantaneously; the liquid disperses amongst neighboring alveoli. That is, the liquid from alveoli that “clear” is in fact more equitably redistributed amongst surrounding alveoli. Referring to FIG. 3, a pair of micrographs depict the spontaneous clearance of a flooded alveolus, indicated by an asterisk (*). The micrographs are sequential optical sections from a z-stack of mages, with a time of about 5 sec between images. In between imaging the two sections, the liquid cleared from the (*)alveolus, leaving it aerated. Hooded alveoli are occasionally seen to clear spontaneously. Alveoli “pop” open as liquid disperses to nearby alveoli. In FIG. 3, the lightly-stippled areas 34 represent the LLL or the alveolar wall.

The stability of flooded alveoli can be understood with the novel analysis of the spatial variation in liquid phase pressure of the flooded alveolus 10 discussed above with respect to FIG. 1. The analysis demonstrates that ventilation over-expands aerated alveoli located adjacent to flooded alveoli and the pressure barrier ΔP_{BARRIER}—equal to P_{LIQ.BORD} at the border between two alveoli minus P_{LIQ.EDEM} within the flooded alveolus-opposes the escape of liquid from discrete, flooded alveoli. To protect against ventilator-induced lung injury, the various aspects of the present invention provide approaches to reduce alveolar over-expansion by overcoming ΔP_{BARRIER} to promote equitable flooding liquid distribution amongst alveoli. Such approaches include, but are not necessarily limited to:
1. Acceleration of deflation during mechanical ventilation, in combination with maintenance of zero end-expiratory pressure (ZEEP) or PEEP, to transiently increase P_{LIQ.EDEM}, reduce the pressure barrier and clear flooded alveoli; and
2. Vibration or step or impulse force application to the lung, which includes vibrating the lung or applying a step or impulse force to the lung to impose spatial variation in surface tension and/or to perturb the normal pressure gradient in the flooded alveolar liquid, and, in a random fashion, increase the likelihood of overcoming the pressure barrier to clear flooded alveoli.

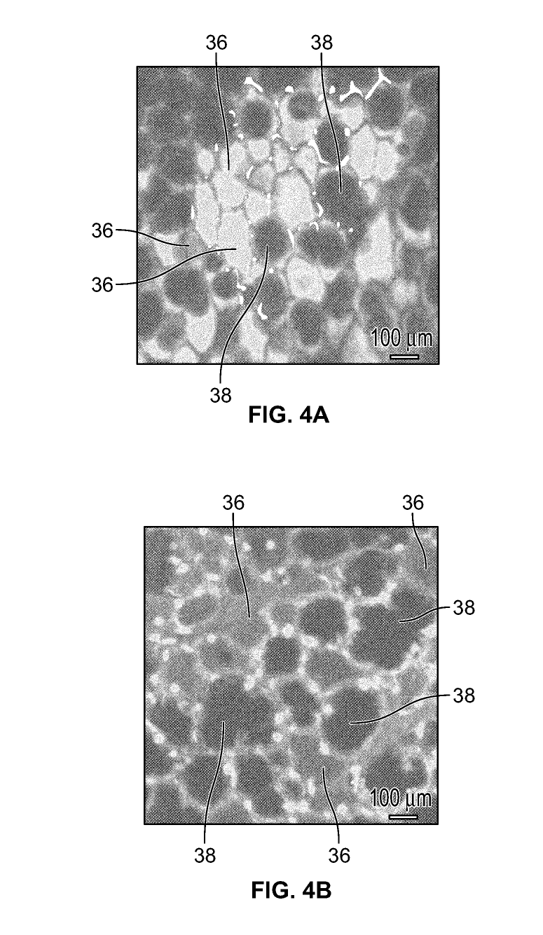

FIGS. 4A and 4B are a pair of micrographs showing alveolar edema models used to explore the efficacy of the foregoing approaches. The model of FIG. 4A is a local model generated by alveolar microinfusion of 5% albumin solution labeled with 2′,7′-bis-(2-carboxyethyl)-5-(and-6)-carboxyfluorescein (BCECF). The model of FIG. 4B is a global permeability edema model, generated by inclusion of 6 millimolar oleic acid in lung perfusate, plus 34 μM fluorescein also in the perfusate for visualization. As shown by comparison of FIGS. 4A and 4B, either method generates the characteristic pattern of interspersed aerated and flooded alveoli. Light and medium gray areas, such as areas 36, indicate the presence of liquid. Darker areas, such as areas 38, indicate aerated alveoli.

### 1. Accelerated Deflation During Mechanical Ventilation

According to embodiments of the present invention, accelerated deflation of the lung will, effectively, catapult flooding liquid out of the alveoli in which it is trapped. Accelerated deflation, as used herein, means sudden or abrupt deflation of the lung, without or with application of vacuum pressure, as described in further detail hereinafter. As also discussed further herein, the effectiveness of accelerated deflation of a lung having flooded alveoli has been demonstrated in the local alveolar edema model and global permeability edema model in the isolated, perfused rat lung.

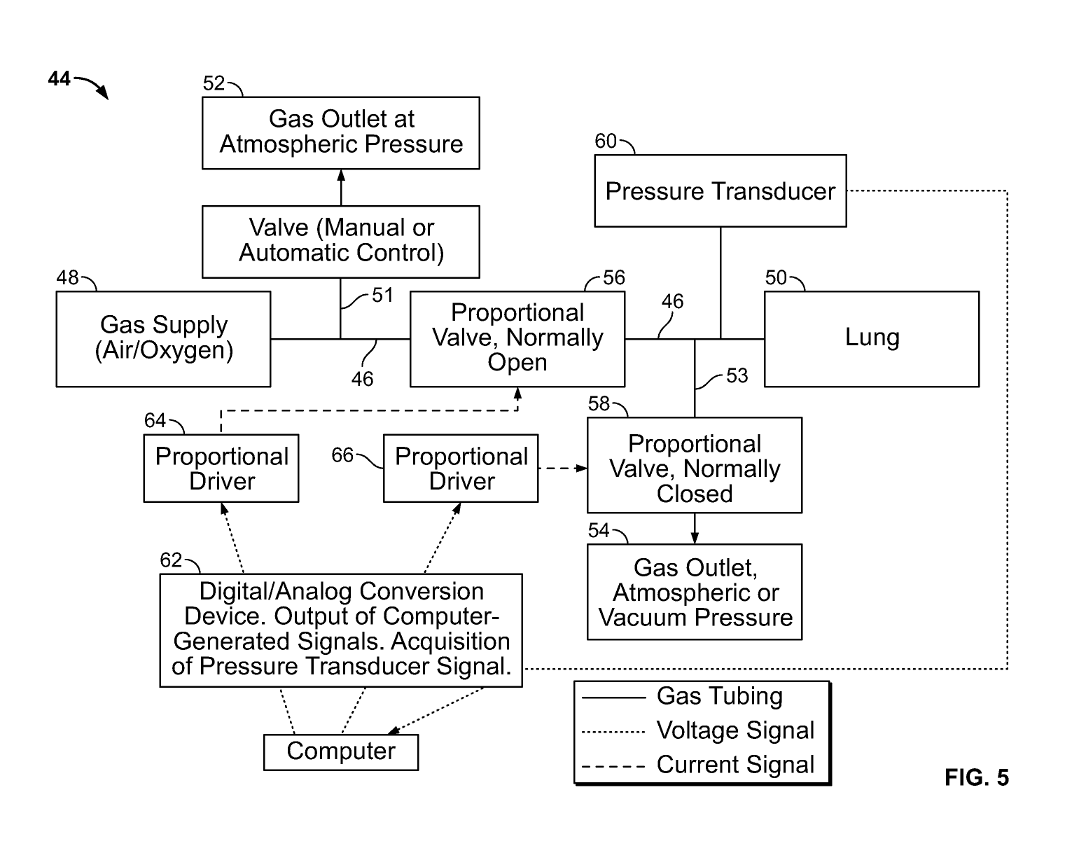

FIG. 5 is a schematic block diagram of an apparatus 44 for the generation of custom ventilation pressure waveforms, according to an embodiment of the present invention. A tubing line 46 links the ventilation gas source 48 to the lung 50. Along the tubing line 46 are two branches 51, 53 to outlets 52, 54, respectively, one outlet 52 opening to atmospheric pressure, the other outlet 54 opening to atmospheric or vacuum pressure. Located along the tubing line 46 between the branches 51, 53 to outlets 52, 54 is a normally-open inflation proportional valve 56, and along the branch 53 to outlet 54 (hereinafter, the ‘deflation branch’) is a normally-closed deflation proportional valve 58. It is noted that, as will be recognized by persons of ordinary skill in the relevant art, the inflation valve may be normally-open or normally-closed and the deflation valve, likewise, may be normally-open or normally-closed, according to the preference of the user. Along the tubing line 46 between the deflation branch 53 to outlet 54 and the lung 50, a pressure transducer 60 measures and indicates pressure in the tubing line 46. The pressure transducer may be located in line with tubing line 46 or attached to tubing line 46 by a connector, depending upon the particular configuration of components, as will be understood by persons of ordinary skill in the relevant art. In some embodiments of the present invention, the pressure transducer 60 is proximate the end of the tubing line 46 where the tubing line 46 is fluidly connected to the lung 50, such that the pressure measured by the pressure transducer 60 is substantially the same as the pressure at the entrance to the trachea (not shown). A custom Labview® program acquires pressure data from the transducer 60 and, in an open-loop fashion, provides voltage signals that control the proportional valves via a digital/analog conversion device 62 and appropriate proportional drivers 64, 66. The development of suitable computer programs and selection of conversion devices 62, drivers 64, 66 proportional valves 56, 58 and pressure transducer 60 are within the ability of those having ordinary skill in the relevant art. In some embodiments of the present invention, the branch 51 to the first outlet 52 is omitted, and the normally-open inflation proportional valve 56 is placed in the tubing line 46 between the end of the tubing line 46 that receives gas from the ventilation gas source 48 and the deflation branch 53 to outlet 54. Also in some embodiments of the present invention, a pneumotachometer for measuring air flow rate is placed along the tubing line 46 between the branch 53 to outlet 54 and the lung 50; the pneumotachometer signal is recorded, through data acquisition device 62, by the computer running Labview, and the Labview program integrates inspiratory or expiratory flow rate to determine tidal volume.

In an embodiment of the present invention, the lung 50 is inflated to a target peak pressure or by a target tidal volume, as follows. Prior to inflation, a maximal voltage signal is being sent to inflation valve 56, such that valve 56 is closed. Subsequently, the voltage signal to valve 56 is decreased gradually, thus opening inflation valve 56 and allowing ventilation gas through inflation valve 56 to inflate the lung. Once the lung is inflated to the target peak pressure or by the target tidal volume, the voltage to inflation valve 56 is returned to the maximum setting to close inflation valve 56. Throughout inflation, 0 volts (V) are sent to deflation valve 58 such that deflation valve 58 is closed. At the end of inflation of the lung 50, there may be a period during which maximum voltage is sent to inflation valve 56 and zero volts are sent to deflation valve 58, such that both valves 56, 58 are closed and the lung is held at constant, peak volume.

The lung 50 is deflated to target minimum pressure or by target tidal volume by increasing voltage to, thus opening, deflation valve 58. The lung may be deflated in various ways, including the following three. 1. The deflation valve 58 may be opened gradually and with atmospheric pressure applied at its outlet 54, to deflate the lung 50 gradually. This option may be employed to ventilate the lung 50 with a sinusoidal pressure waveform. This is passive, unaccelerated deflation. 2. The deflation valve 58 may be opened suddenly, by application of a step voltage increase, and with atmospheric pressure applied at its outlet 54, to deflate the lung 50 passively but suddenly. This option may be employed to ventilate the lung 50 with a sawtooth waveform in which deflation is passively accelerated. 3. The deflation valve 58 may be opened suddenly, by application of a step voltage increase, and with vacuum pressure applied at its outlet 54, to actively accelerate lung deflation. This option may be employed to ventilate the lung 50 with an accelerated sawtooth waveform in which deflation is actively accelerated. It should be noted that the resistance to expiratory air flow of the outflow path of the ventilation circuit—the path from the lung 50 through the portion of tubing line 46 between the lung 50 and the deflation branch and through the deflation branch to outlet 54—contributes to lung deflation rate such that when outflow path resistance is low even passively accelerated deflation may have a high deflation rate.

Regardless of deflation method, valve 58 remains open until the pressure measured in the tubing line 46 has decreased to a targeted pressure, which may be zero or positive, for ZEEP or PEEP ventilation, respectively, at which time voltage to deflation valve 58 is returned to zero, causing deflation valve 58 to close. The return of the voltage to deflation valve 58 to zero may be accomplished with a step voltage decrease at the end of deflation or with a gradual decrease in the later portion of the deflation period in order to avoid oscillations in the ventilation pressure waveform. Throughout deflation, maximal voltage is sent to inflation valve 56 such that inflation valve 56 is closed.

Thus, the lung 50 may be deflated with passive or active acceleration while maintaining a zero (ZEEP) or positive (PEEP) pressure at the lung 50 at the end of expiration. This maintenance of ZEEP or PEEP is one of the characteristics of the present invention that distinguishes it over methods existing in the prior art.

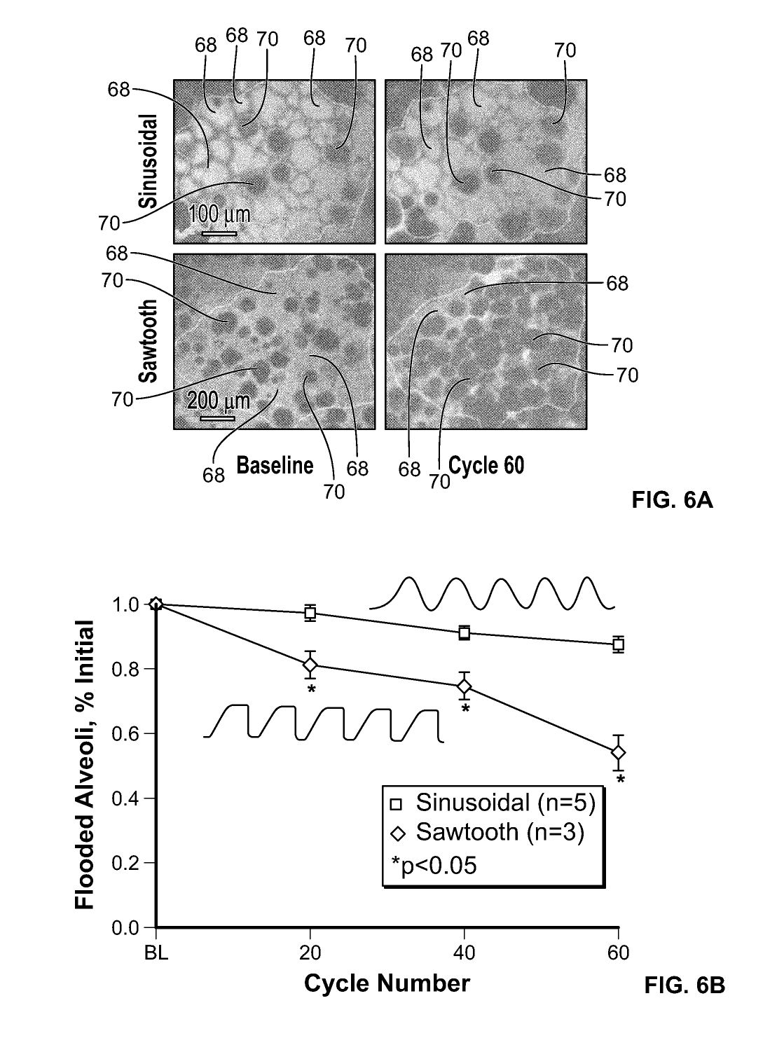

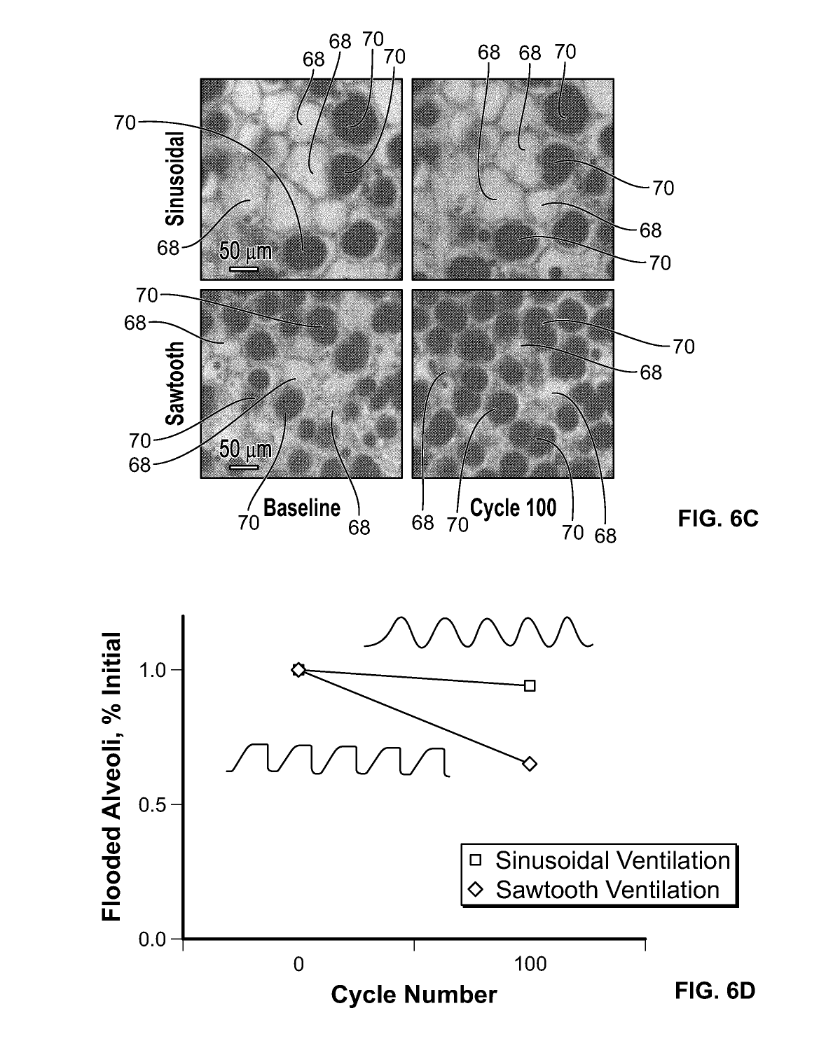

FIGS. 6A and 6B are a pairing of a set of micrographs and a graph comparing clearance of alveoli by ventilation using sinusoidal and sawtooth pressure waveforms, as are FIGS. 6C and 6D. FIGS. 6A and 6B illustrate results obtained using a local edema model, and FIGS. 6C and 6D illustrate results obtained using a global permeability edema model. Both ventilation patterns were used at a cycle frequency of 0.2 Hz between P_{AW} of 5 and 15 cmH₂O. Baseline (BL) in FIGS. 6A and 6B is following 20 cycles of sinusoidal ventilation in each group, to clear unstably flooded alveoli and test the ventilation patterns on stably flooded alveoli. As can be seen from the micrographs and graphs of FIGS. 6A, 6B, 6C, and 6D, ventilation using a sawtooth waveform clears a greater number of alveoli than does ventilation using a sinusoidal waveform, indicating that the abrupt deflation of the sawtooth ventilation clears alveolar liquid more effectively than sinusoidal ventilation. In FIGS. 6A and 6C, exemplary flooded alveoli 68 are indicated by lighter gray areas, and exemplary aerated alveoli 70 are indicated by darker areas.

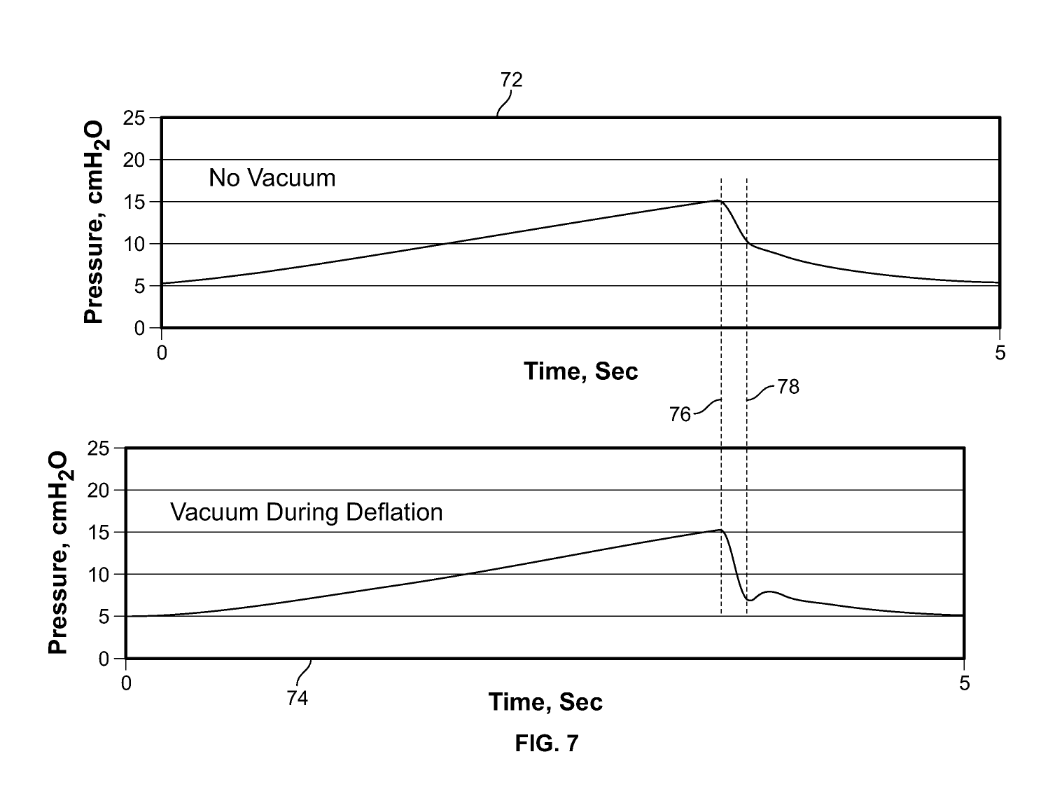

In some embodiments of the present invention, lung deflation may be actively accelerated (accelerated sawtooth), by applying vacuum pressure at gas outlet 54 of the ventilation apparatus 44 shown in FIG. 5 and opening valve 58 suddenly at the start of deflation. The effect of this actively accelerated deflation is shown in the graphs of FIG. 7. The upper graph 72 shows a waveform generated with atmospheric pressure at outlet 54 and sudden opening of valve 58 at the start of deflation. As the resistance of the outflow path of the ventilation circuit in this embodiment was relatively low, the peak deflation rate, even in the absence of active acceleration, was a relatively high 33 cmH₂O/sec. Nonetheless, vacuum application further accelerated deflation. The lower graph 74 shows a waveform generated with vacuum pressure applied at outlet 54 and sudden opening of valve 58 at the start of deflation. The vertical lines 76, 78 indicate the time for the waveform of the upper graph (i.e., the waveform generated without vacuum application) to decrease from 15 to 10 cmH₂O. As shown in the lower graph, application of vacuum at outlet 54 generates a waveform having a sharper deflation slope, with a shorter time required for pressure to decrease from 15 to 10 cmH₂O. At the same time as deflation was accelerated, PEEP was maintained (i.e., tracheal pressure never decreased below a set, positive threshold value).

As discussed above with respect to FIGS. 5, 6A, 6B, 6C, 6D, and 7, accelerated deflation of the lung is effective in clearing liquid from alveoli. Such clearance may be achieved with one or a combination of the following methods, performed according to embodiments of the present invention:
1. Allowing sudden escape of air from the lung such as is accomplished by sudden opening of a valve along the deflation outlet branch of the ventilation tubing circuit (e.g., normally closed valve 58 at the gas outlet 54 in the apparatus of FIG. 5) during deflation;
2. Applying vacuum pressure at the exit of the deflation outlet branch of the ventilation tubing circuit (e.g., gas outlet 54 in the apparatus of FIG. 5) during deflation; and
3. Stimulating the abdominal and/or intercostal muscles, by functional electrical stimulation, or other means, to generate a cough-like motion synchronized with exhalation/deflation.

Any of the above methods for causing accelerated deflation, alone or in combination, could be combined with mechanical ventilation; non-invasive ventilation; or lung expansion devices including chest physiotherapy devices and high frequency oscillation devices.

Vacuum may be applied by known means such as vacuum pump, house vacuum line, Venturi tube, reciprocating piston or other mechanism. However, a distinguishing feature of the apparatus of FIG. 5, according to embodiments of the present invention, is the inclusion of a valve along the deflation outlet branch that leads to an atmospheric or vacuum pressure source and regulation of that valve in response to pressure measured near the trachea. Previous ventilators that used Venturi tubes to accelerate deflation reduced tracheal pressure below atmospheric pressure at the end of expiration (i.e., ventilated with NEEP), thus did not maintain either PEEP or ZEEP. The apparatus 44 of the present invention, by applying vacuum pressure at the exit 54 of the deflation branch 53 of the ventilation circuit, and terminating vacuum application when tracheal pressure decreases to the desired PEEP level or to zero, enables deflation to be actively accelerated while maintaining PEEP or ZEEP at the trachea.

### 2. Vibration or Step or Impulse Force Application to the Lung

Lung motion during breathing is normally smooth. Application of vibration or of step or impulse force to the edematous lung could perturb surface tension within flooded alveoli in such a fashion as to facilitate more equitable flooding liquid distribution.

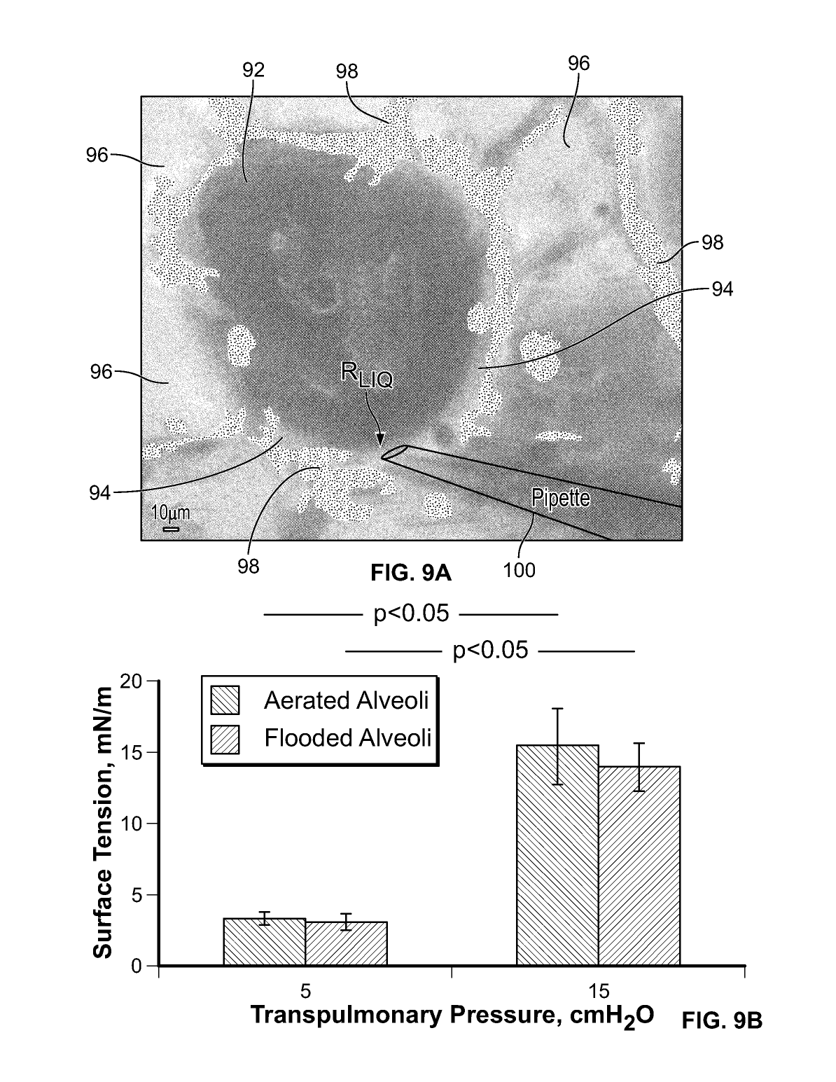

Surface tension is normally spatially uniform in the lung. FIGS. 9A and 9B are a pairing of an enhanced micrograph demonstrating how surface tension is determined in an aerated alveolus and a graph indicating that surface tension is spatially uniform. The micrograph is an image of an aerated alveous 92, with a liquid lining layer 94, surrounded by flooded alveoli 96. Alveolar walls 98 are indicated by light stippling. The pipette measures the liquid lining layer pressure in the aerated alveolus 92 for surface tension determination according to the Laplace relation. The graph presents grouped surface tension data for adjacent aerated and flooded alveoli (n=3), showing that surface tension does not vary spatially even in a region of heterogeneous alveolar flooding.

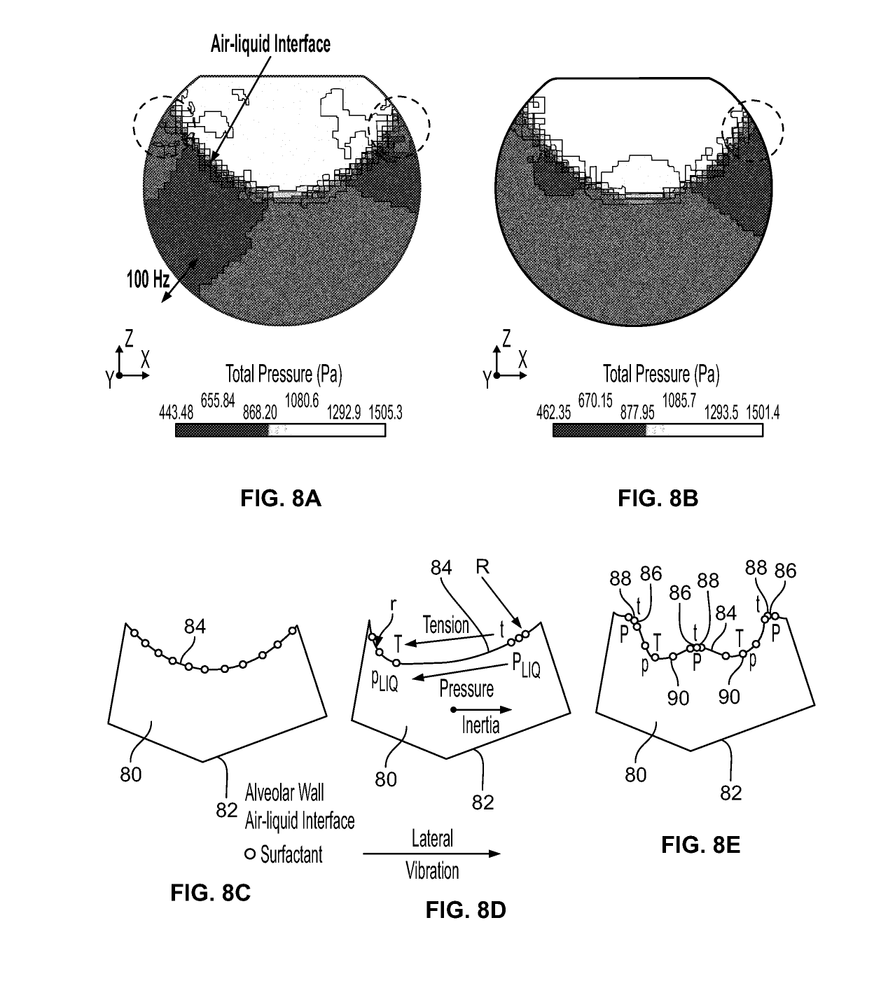

Lung vibration could alter the normally uniform surface tension distribution. FIGS. 8C, 8D, and 8E are a group of schematic drawings indicating a conceptual model of vibration effects on flooded alveolar surface tension. Liquid 80 fills the area between the alveolar wall 82 and the air-liquid interface 84. Referring to FIG. 8C, at a normal breathing frequency (0.2 Hz), surfactant distribution and surface tension are constant along the interface 84. Referring to FIG. 8D, a rightward lateral vibration stroke propels the center of the liquid mass 80 to the right because of inertia, and skews the interface 84 to the right such that the interfacial radius R at the right is greater than the radius r at the left. The movement of the liquid 80 compresses the surfactant and lowers surface tension t at the right, and dilates the surfactant and raises the surface tension T at the left, thus generating a tension force to the left. Due to the Laplace relation, liquid pressure P_{LIQ} at the right is greater than pressure p_{LIQ} at the left, thus a net pressure force also acts to the left. Just as interplay between inertia and pressure can cause a resonant “rocking mode” during vibration of a pure water droplet, interplay between inertia, surface tension and pressure has the capacity to generate a “rocking mode” in a flooded alveolus, as depicted in FIG. 8D. Higher frequency vibration, likewise due to the interplay of inertia, surface tension, and pressure, has the potential to generate resonant capillary waves. Referring to FIG. 8E, such resonant capillary waves 86 would compress the surfactant and lower surface tension at the crests 88 of the waves 86 and dilate the surfactant and raise tension at troughs 90 of the waves 86.

If surface tension gradients existed along the interface 84, however, they would apply shear stress to, and cause movement of, the liquid 80 below the interface 84. Thus, vibration of the lung, or application of a step or impulse force to the lung, would generate surface tension gradients at the air-liquid interface 84, and accompanying pressure gradients in the flooding liquid 80 below the interface 84. Such induced spatial variation in the surface tension or pressure has the potential to overcome, at random, the pressure barrier trapping liquid in discrete alveoli, therefore to promote clearance of flooded alveoli.

Flooded alveolar liquid pressure is normally maximal at the edge of the alveolus. In the flooded alveolus, liquid pressure P_{LIQ.BORD} at the edge of the alveolus exceeds liquid pressure P_{LIQ.EDEM} in the center of the alveolus (see FIG. 1). Between the two locations, pressure may be assumed to vary smoothly, governed by the smooth variation in interfacial curvature. Perturbation of the normal smooth breathing motion, however, might perturb the typical pattern of pressure variation in flooding liquid and cause pressure at the edge of the alveolus transiently to fall below pressure in the center of the alveolus. Referring to FIGS. 8A and 8B, computation fluid dynamics modeling indicates that such a transient reversal of the pressure barrier is possible. FIGS. 8A and 8B show modeling predictions for the effect of lung vibration on flooded alveolar liquid pressure distribution. In the computation fluid dynamic model (Star-CCM+) of FIGS. 8A and 8B, an alveolus is approximated as a 100 micron diameter 3-D sphere with three-quarters of its volume filled with water. Air pressure is modeled at 15 cmH₂O. Surface tension is modeled at 15 mN/m, with liquid slipping at the boundary. In FIGS. 8A and 8B, the simulated pressure increases from the darker shading to the lighter shadings. In the simulation, the alveolus was vibrated at 100 Hz, and a 45 deg angle. The dashed circles highlight pressure at what would be the border with an adjacent alveolus. Liquid pressure is generally highest at the border, as in FIG. 8A, but sometimes decreases, as in FIG. 8B. Thus, the normal pressure distribution might be inverted independent of any perturbation to interfacial curvature or surface tension. Such a reversal of pressure gradient could, transiently, overcome ΔP_{BARRIER} and facilitate clearance of the flooded alveolus.

When vibrating the lung from its periphery, sufficient amplitude is required to overcome damping as the signal propagates. A high frequency signal will travel better through water than air. Thus, the greater the flooding of the lung, the more effective vibration would be as a therapy. In a droplet of pure water as small as an alveolus, the first resonant (rocking) mode would be expected to occur at about 5000 Hz. With the particular geometry of the flooded alveolar interface and inclusion of surfactant at the interface, the resonant frequency is not known, and, in view of the current state of art, is likely to require empirical investigation. But, even non-resonant vibration might alter the normal flooding liquid pressure distribution in a manner that favors alveolar clearance.

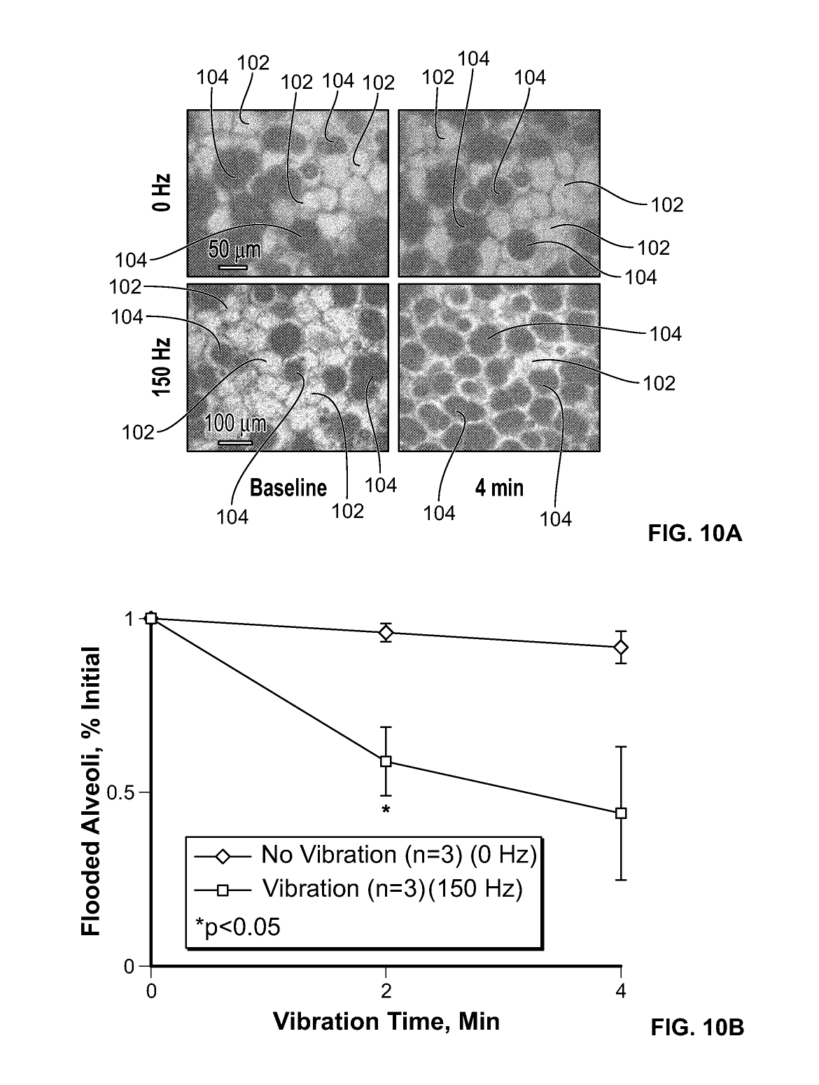

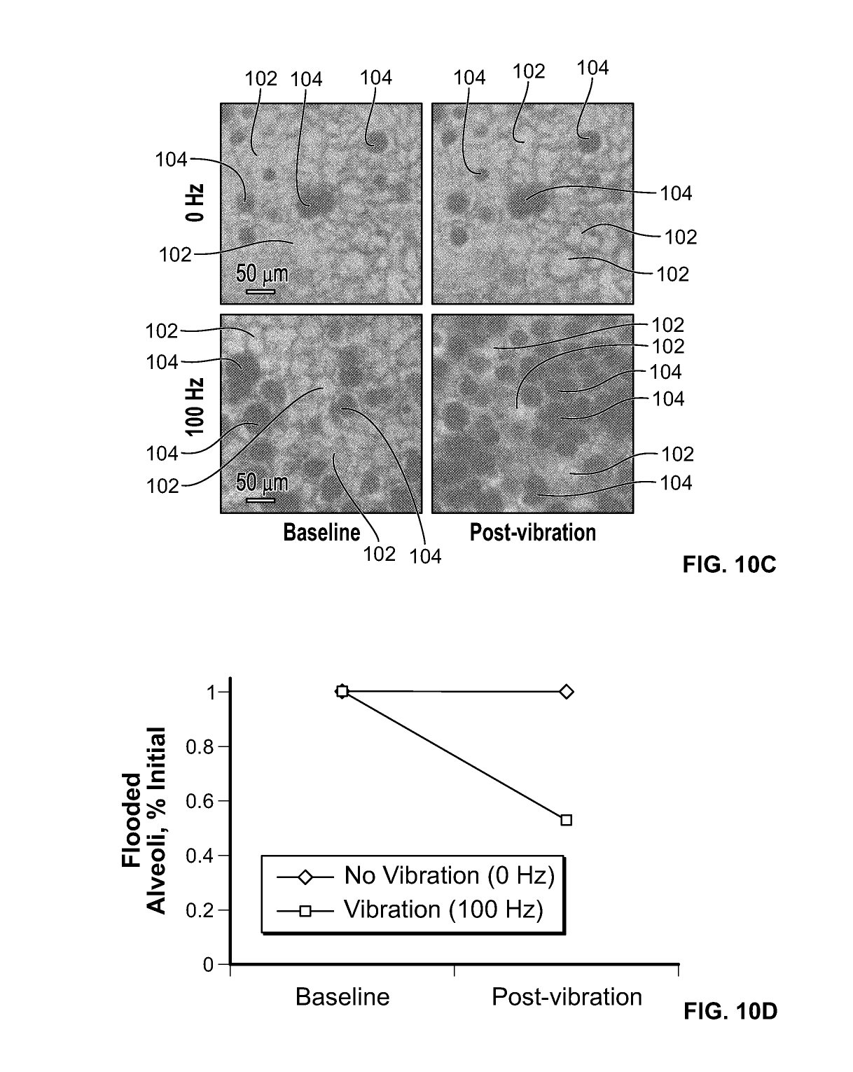

Given the tradeoff between amplitude and frequency, initial tests were performed in the relatively low frequency range of 100-200 Hz. With the local edema model and with the global permeability edema model, vibration of the lung was tested for its ability to clear flooded alveoli. A function generator was used to drive a speaker coil and the speaker cone was placed in contact with the lung surface, separated from the lung by saran wrap. As a control, the speaker cone was pressed against the lung surface with the same force, but in the absence of power to the speaker, such that the speaker cone did not vibrate. As discussed below with relation to FIGS. 10A, 10B, 10C, and 10D, vibration was found to redistribute flooding liquid more equitably in both edema models.

FIGS. 10A and 10B are a pairing of a set of micrographs with a graph indicating that that vibration of the lung surface promotes alveolar liquid clearance, as are FIGS. 10C and 10D. FIGS. 10A and 10B show vibration results in the presence of a local edema model. The flooding liquid is 5% albumin in normal saline with 32 μM BCECF. To clear unstable alveoli, the lung is ventilated with 20 sinusoidal cycles between 5 and 15 cmH₂O at 0.2 Hz prior to baseline (cycle 0). The micrographs of FIG. 10A include images of the flooded area at baseline and after four minutes of being pressed against a speaker coil (separated by saran wrap) while the speaker is unpowered (control) or vibrating at 150 hertz (Hz) (vibration), The lung was constantly inflated at P_{AW} of 15 cmH₂O during the experiment. It can be seen that vibration effectively cleared the alveoli. FIG. 10B presents grouped results graphically. FIGS. 10C and 10D present the results of the same experiment as that of FIGS. 10A and 10B, replicated in a global permeability edema model with fluorescein (36 μM) included in the perfusate. The lung was vibrated at 100 Hz for 2 min, while held at constant P_{AW} of 15 cmH₂O. It can be seen that vibration effectively cleared the alveoli in this model also. In FIGS. 10A and 10C, flooded alveoli 102 are shown as light or medium gray areas, and aerated alveoli 104 are shown as darker areas 104.

To apply vibrations of ≧50 Hz to the lung for edema clearance, the following methods could be employed individually, in combination and/or in conjunction with mechanical ventilation; non-invasive ventilation; or lung expansion devices including chest physiotherapy devices and high frequency oscillation devices, according to various embodiments of the present invention:
1. Coupling a speaker coil, oscillator or ultrasound generator to the patient's chest wall or back;
2. Implanting a speaker coil, oscillator or ultrasound generator in the fluid-filled plural space (outside the lungs, inside the ribcage);
3. Inserting a fluid-filled conduit into the pleural space and, via the conduit, hydraulically applying a high frequency pressure signal to the pleural fluid, with, e.g., a speaker coil, oscillator or an ultrasound generator;
4. Coupling a speaker coil, oscillator or ultrasound generator to the trachea, either directly or through the skin;
5. Percussing the chest and/or back with a commercially-available device intended for that purpose (e.g., a pneumatic vest); and
6. Adding a ≧50 Hz component to an existing ventilation pressure, volume or flow waveform.

In some embodiments of the invention, a step or impulse force could be applied to the lung, rather than a vibration. In ideal form, step and impulse functions are of infinite frequency. The actual frequency of force application to the lung would not be infinite, but would be maximal. Thus, repetitive application of a step or impulse force to the lung would promote flooded alveolar clearance. A step or impulse function would be employed alone or in conjunction with mechanical ventilation; non-invasive ventilation; or lung expansion devices including chest physiotherapy devices and high frequency oscillation devices, by one of the following methods:
1. Any of the mechanisms discussed above with respect to vibration of the lung at high frequency;
2. Any of the mechanisms for sudden deflation discussed in Section 1;
3. Transient airway occlusion during deflation, particularly in combination with active, accelerated deflation. Transient airway occlusion could be effected with a valve that is transiently closed, or with a spinning ball or high frequency flow interrupter, such as are used in high frequency percussive ventilation, along the outflow path of the ventilation circuit; or with another mechanism. Deflation could be accelerated by any of the mechanisms discussed in Section 1; by use of a Hayek Oscillator; or by other means.

It will be understood that the embodiments described herein are merely exemplary and that a person skilled in the art may make many variations and modifications without departing from the spirit and scope of the invention. All such variations and modifications are intended to be included within the scope of the invention described in the claims appended hereto.

### Example

In isolated, perfused rat lungs, it was investigated whether acceleration of deflation with application of vacuum pressure during mechanical ventilation could promote liquid escape from flooded alveoli.

### Materials and Methods

Isolated, Perfused Lung Preparation.

All animals were handled in accord with a protocol approved by the Stevens Institute of Technology Institutional Animal Care and Use Committee. Male Sprague-Dawley rats (n=20, 320-340 g) were anesthetized (2.5-4% isoflurane in 100% oxygen) and the isolated, perfused rat lung was prepared as detailed previously (see, Wu Y, Perlman C E, In situ methods for assessing alveolar mechanics, J Appl Physiol 112: 519-526, 2012, which is hereby incorporated by reference herein). The lungs were positioned with the costal surface upward; pulmonary arterial and left atrial cannulas were connected to a perfusion circuit; and the lungs were initially inflated P_{AW} of 30 cmH₂O. P_{AW} was then reduced to a constant baseline value of 5 cmH₂O and the isolated lungs were perfused with 10 ml of autologous blood plus 18 ml of 5% bovine serum albumin (commercially available from Sigma Aldrich located in St. Louis, Mo., USA) in normal saline at 12 ml/min and 37° C. Left atrial pressure was set to 3 cmH₂O and pulmonary arterial pressure was 10 cmH₂O.

Lung Ventilation.

The lungs were ventilated with a specified PEEP and V_{T} in the absence or presence of accelerated deflation.

A custom-designed ventilation circuit was used (FIG. 5). Pressure regulators were used to control pressure at the inlet to the circuit, such that drift was less than 0.05 cmH₂O/min (see, Kharge A B, Wu Y, Perlman C E, Surface tension in situ in flooded alveolus unaltered by albumin, J Appl Physiol 117: 440-451, 2014, which is hereby incorporated by reference herein). Most of the air flowed out of a bleed valve on an outlet branch at the start of the air-flow line to the lungs. Progressing along the air-flow line to the lungs were, in sequence, a normally-open proportional valve (obtained from Parker-Hannifin located in Fairfield, N.J., USA) that controlled lung inflation; a second outlet branch with a normally-closed proportional valve (obtained from Clippard located in Cincinnati, Ohio, USA) that controlled lung deflation; and a blind-ended branch, located just proximal to the tracheal cannula, leading to a pressure transducer that recorded P_{AW}. A custom Labview program sent voltages out of two channels of a data acquisition device (Model USB-6259 by National Instruments located in Austin, Tex., USA) to two proportional controllers (obtained from Canfield Connector located in Youngstown, Ohio, USA). The proportional controllers converted the voltages to currents and sent the latter to the proportional valves. The same Labview program recorded P_{AW}, acquired through the same data acquisition device.

Based on a calibration previously reported in Wu Y, Kharge A B, Perlman C E, Lung ventilation injures areas with discrete alveolar flooding, in a surface tension-dependent fashion, J Appl Physiol 117: 788-796, 2014, which is hereby incorporated by reference herein, the lung was ventilated between specified minimal and maximal P_{AW} values to mimic ventilation with a given PEEP and V_{T}. At the start of inflation, a maximal 5 V signal was sent to the inflation valve and a minimal 0 V signal was sent to the deflation valve, such that both valves were closed. During inflation, voltage to the inflation valve was decreased in an exponential fashion such that P_{AW} rise exponentially with time. When P_{AW} plateaued at the target maximal value, P_{AW.MAX}, 5V were sent to the inflation valve to close the valve and stop inflation. At the start of deflation, a maximal 5V were sent to the deflation valve to fully open the deflation valve. Over the course of deflation, voltage to the deflation valve was reduced in a stepwise fashion until P_{AW} plateaued at the target minimal value. At this point, voltage to the deflation valve was returned to zero such that the valve closed. With such gradual closure of the deflation valve, P_{AW} did not oscillate at the end of deflation. When maximal voltage was sent to the inflation valve and no voltage to the deflation valve, the lungs were held at constant inflation.

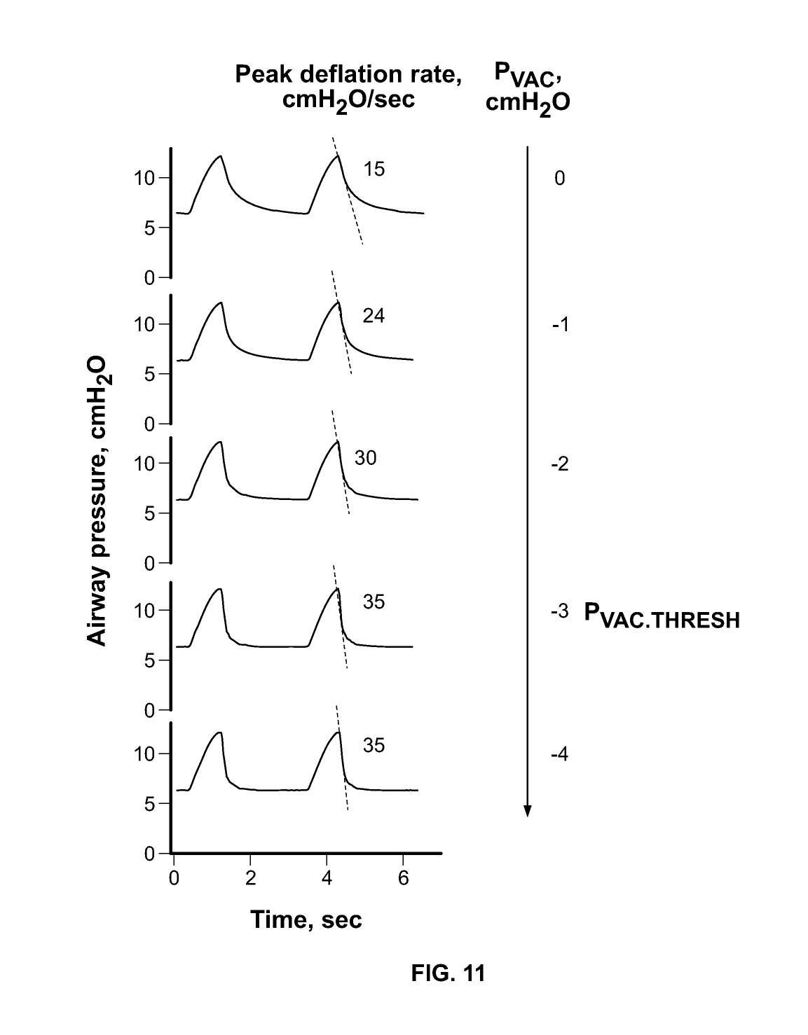

With this apparatus, deflation could, optionally, be actively accelerated by applying a vacuum pressure to the outlet of the deflation valve. Even with vacuum application, ZEEP or a targeted PEEP was always maintained. As there is a limit to the degree to which deflation can be accelerated, for each lung used the application of increasing vacuum pressure, in 1 cmH₂O increments, was initially tested to determine the threshold vacuum pressure, P_{VAC.THRESH}, beyond which a further decrease in P_{VAC} caused no further acceleration of deflation (FIG. 11). Deflation in the experiment was actively accelerated using a P_{VAC} that was 1 cmH₂O more negative than P_{VAC.THRESH}.

While ventilating the lungs, P_{AW} was recorded with a sampling frequency of 25 Hz. From the recorded pressure traces, deflation rate was quantified at each time point during deflation as the average deflation rate over the two sampling periods bracketing the time point. From these data, the peak deflation rate for each combination of ventilation settings was identified.

Edema Model.

To generate a local model of alveolar edema, an alveolus on the costal surface of the lung was punctured with a glass micropipette (oval tip opening with 4 micrometer (μm) minor diameter, 12 μm major diameter). Through the micropipette were instilled approximately 300 nanoliters normal saline solution containing 3% albumin and labeled with 23 μM fluorescein, which does not alter T (see, Kharge 2014), for visualization. The fluid flooded essentially all regional alveoli and then cleared from some, leaving behind a heterogeneously flooded field (see, Wu 2014).

Imaging.

At baseline and during pauses in ventilation, an O-ring support was used to lower a cover slip just in contact with the costal surface of the lungs (see, Wu 2012). The O-ring and cover slip were used to support a saline drop into which an ×40 water immersion objective (0.8 numerical aperture, 3 millimeters working distance, apochromatic) was lowered for fluorescent confocal imaging (confocal model TCS SP5 from Leica Microsystems located in Buffalo Grove, Ill., USA). Fluorescein fluorescence was excited at 488 nanometers (nm) and emitted light collected above 493 nm. Optical sections that were 369 micromolar (μm) square (1024 pixel square) and 2 μm thick were collected at a subpleural depth of 20 μm.

Experimental Protocol.

The effect of accelerated deflation on flooded alveolar clearance was assessed as follows. After generating a local edema model, the region was imaged at a constant P_{AW} of 5 cmH₂O at baseline. Then the O-ring and cover slip were removed and the lungs were provided with 300 ventilation cycles with a PEEP of 0, 5 or 15 cmH₂O, a V_{T} of 6 or 12 milliliters/kilogram body weight (ml/kg) and a frequency of 0.33 Hz, without or with actively accelerated deflation (i.e., deflation assisted with application of vacuum pressure). After 30, 100, 200 and 300 cycles, ventilation was paused, the lungs were held at constant P_{AW} of 5 cmH₂O, the O-ring and cover slip were temporarily re-positioned in contact with the costal surface and the area was re-imaged.

Analysis of Alveolar Flooding Distribution.

Alveolar flooding distribution was analyzed in a set of alveoli that were present in the confocal images from all time points, using Image J (National Institutes of Health, Bethesda, Md.). Clearance was assessed in two ways. First, the percentage of alveoli in the analysis set that were flooded was quantified; a decrease in % flooded alveoli over time indicated clearance. Second, a ‘% clearance’ metric was calculated for the final time point as (number of flooded alveoli at baseline−number of flooded alveoli at final time point)/(number of flooded alveoli at baseline). Further, noting that a septum can separate two aerated alveoli, two flooded alveoli or an aerated alveolus and a flooded alveolus, and that the last group are over-distended by the pressure difference P_{ALV}−P_{LIQ.EDEM} (see FIG. 1 and also Perlman C E, Lederer D J, Bhattacharya J, Micromechanics of alveolar edema, Am J Respir Cell Mol Biol 44: 34-39, 2011), ‘flooding heterogeneity’ was quantified as (number of septa separating aerated and flooded alveoli)/(total number of septa).

Statistics.

Data are reported as mean±standard deviation. Statistical comparisons between groups were made by ANOVA and post hoc Tukey's analysis. Statistical differences were accepted at p<0.05. When comparing responses to three independent parameters—PEEP, V_{T}, and absence/presence of actively accelerated deflation—differences were assessed between groups for which only one independent parameter differed.

### Results

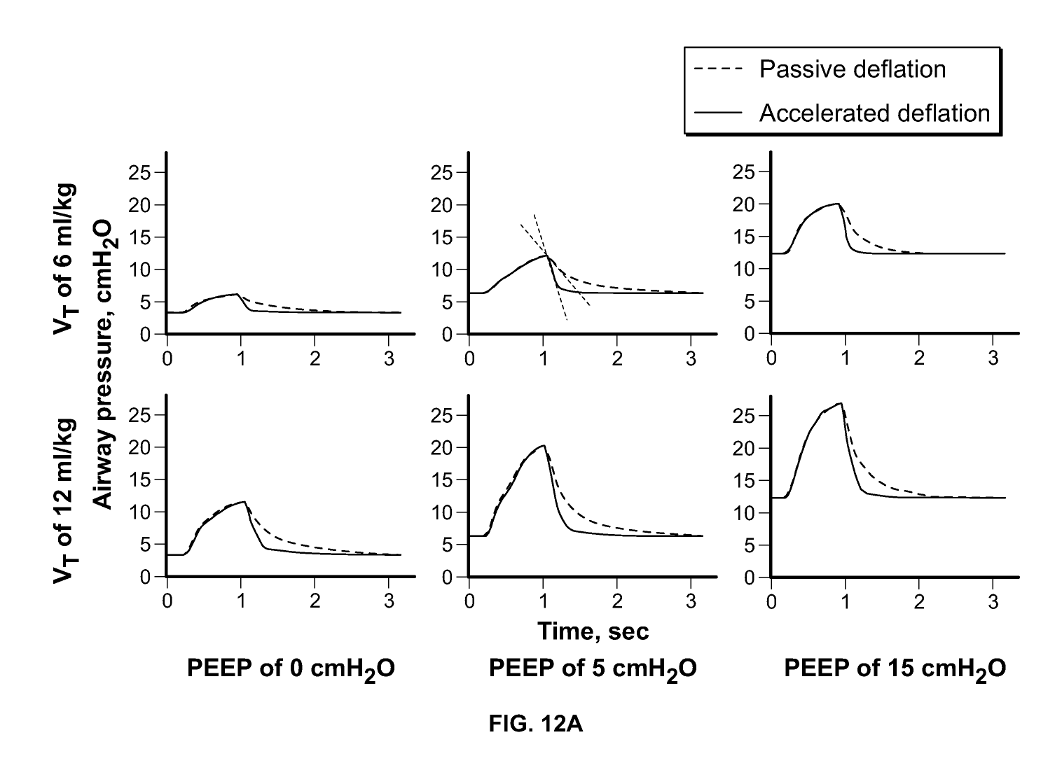

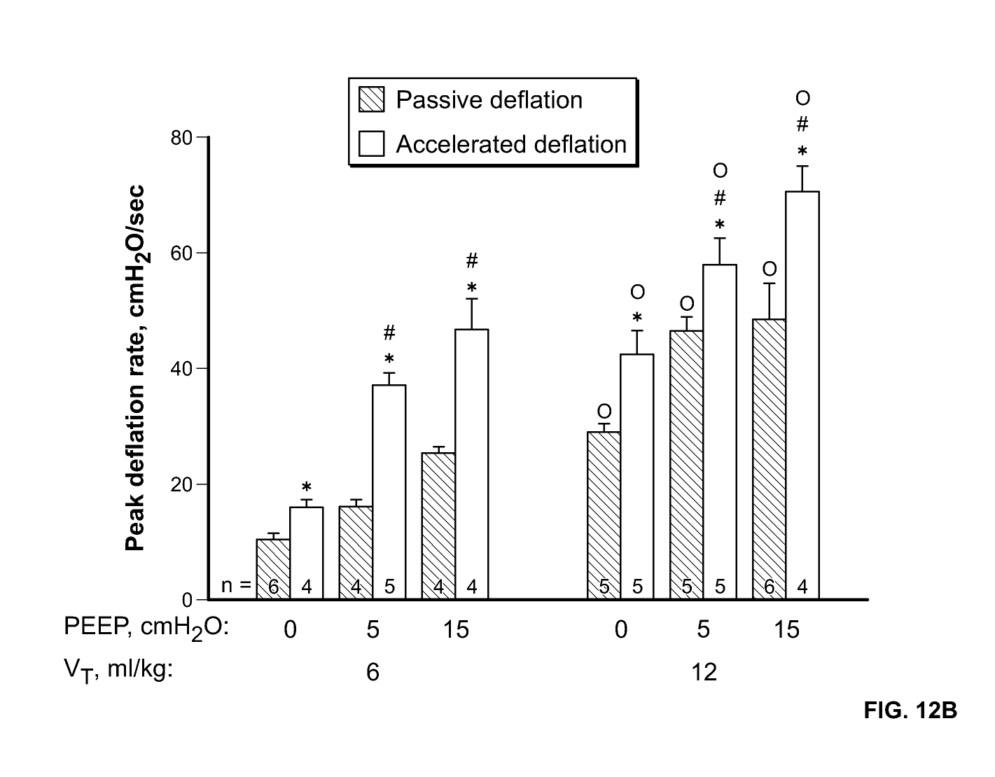

The effect of ventilation conditions on ventilation pressure trace is shown in FIG. 12A and grouped data for peak deflation rate are shown in FIG. 12B. In the absence or presence of vacuum application, increasing PEEP or V_{T} increases the peak deflation rate. For any combination of PEEP and V_{T}, vacuum application increases peak deflation rate. Vacuum application also decreased the time from the start of deflation to the occurrence of peak deflation rate from 0.04-0.12 seconds (sec) to 0.04-0.08 sec.

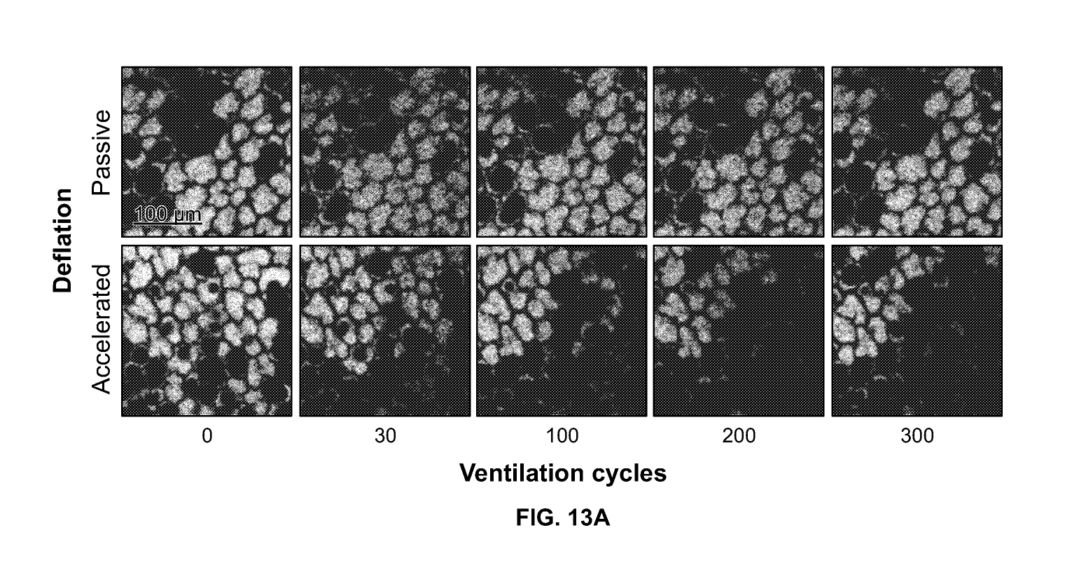

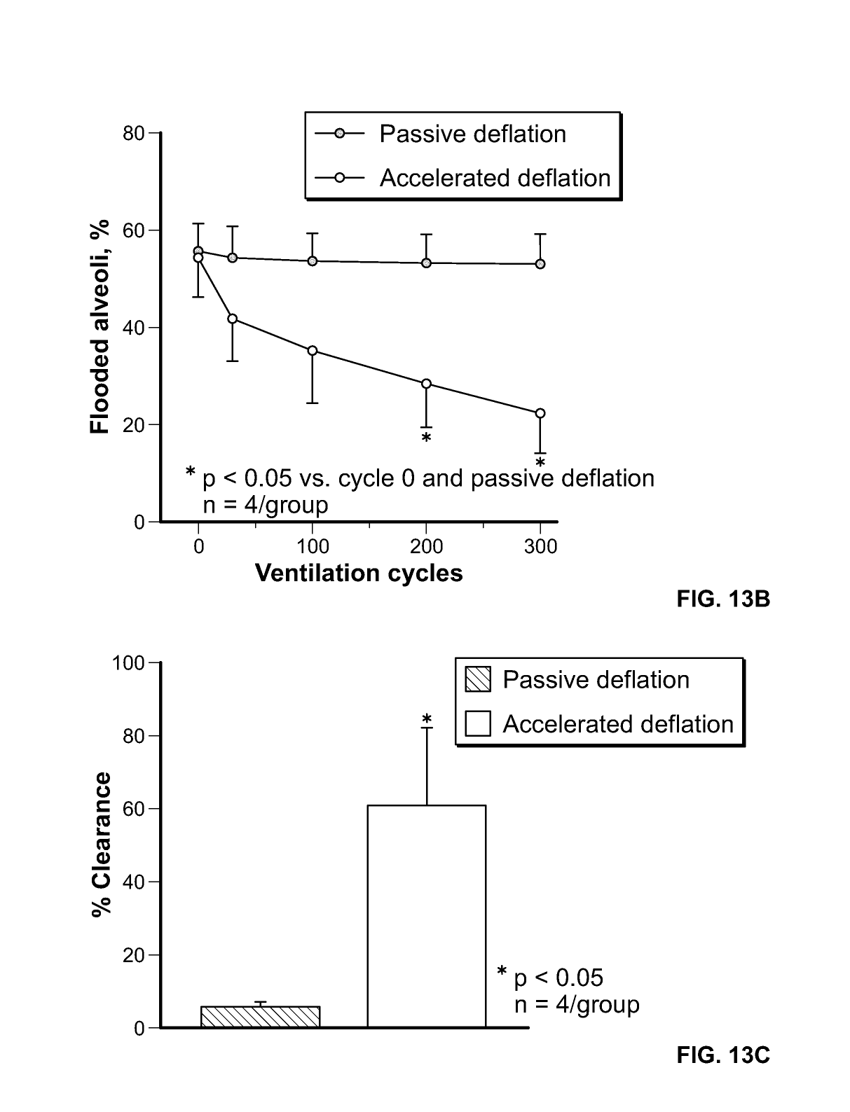

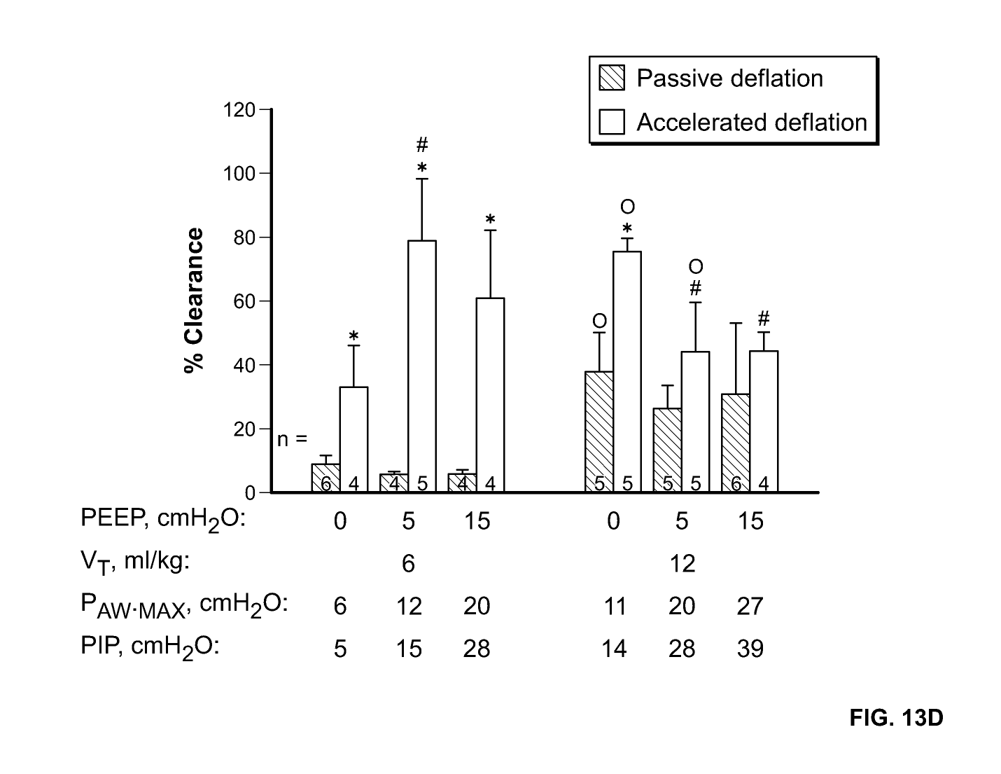

To investigate the effect of accelerated deflation on alveolar clearance, the fraction of flooded alveoli was quantified in fluorescent images of the lung surface. At baseline in the local edema model, 53±7% (n=8) of alveoli were flooded. In the absence of accelerated deflation, ventilation with PEEP of 15 cmH₂O and V_{T} of 6 ml/kg failed to reduce the fraction of flooded alveoli (FIGS. 13A and 13B). With accelerated deflation, 300 ventilation cycles with the same PEEP and V_{T} decreased the fraction of flooded alveoli to 22±8% (FIGS. 13A and 13B), thus causing 60±6% clearance (FIG. 13C).

With passive deflation (step opening of deflation valve without vacuum application, to achieve sawtooth ventilation), but not accelerated deflation (step opening of deflation valve with vacuum application, to achieve actively accelerated sawtooth ventilation), the trend in clearance (FIG. 13D) approximated that in peak deflation rate (FIG. 12B). With passive deflation clearance, like peak deflation rate, was greater at higher V_{T}. The similarity in clearance and peak deflation rate trends may be attributable to the higher lung recoil at P_{AW.MAX} with higher V_{T}. (The values of peak inspiratory pressure (PIP) in a closed-chest scenario that correspond to particular P_{AW.MAX} values in an isolated lung preparation are listed in FIG. 13D.) With accelerated deflation, clearance did not follow the same trend as peak deflation rate but, rather, was greatest with a moderate P_{AW.MAX} of 12 cmH₂O, equivalent to a peak inspiratory pressure of 15 cmH₂O in a closed-chest scenario (see, e.g., Pelosi P, Goldner M, McKibben A, Adams A, Eccher G, Caironi P, Losappio S, Gattinoni L, Marini J J, Recruitment and derecruitment during acute respiratory failure: an experimental study, Am J Respir Crit Care Med 164: 122-130, 2001, which is hereby incorporated by reference herein, and Wu 2014).

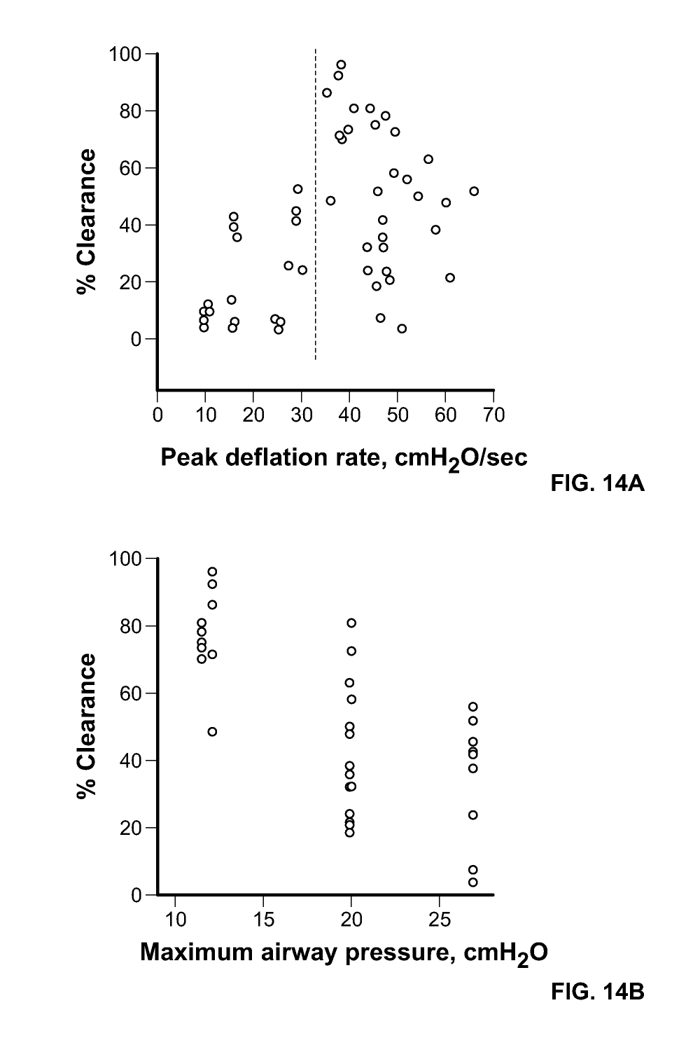

The relation between clearance and peak deflation rate is shown in FIG. 14A and it is apparent that the highest clearance occurred only for peak deflation rates in excess of 33 cmH₂O/sec. The deflation rate of 33 cmH₂O/sec appeared to constitute a threshold required to achieve high clearance. However, a peak deflation rate above this threshold did not guarantee high clearance. The subset of data for which peak deflation rate was greater than 33 cmH₂O/sec is shown in FIG. 14B. It was apparent that, in addition to a high peak deflation rate, a low P_{AW.MAX} was required to promote high clearance. Without intending to be limited by theory, it is believed that a low P_{AW.MAX} maintains a low maximal surface tension at the start of deflation, which ensures a low pressure barrier at the start of deflation. Thus a low P_{AW.MAX}, by keeping ΔP_{BARRIER} low, may enable a high peak deflation rate to overcome ΔP_{BARRIER} and achieve alveolar clearance.

The effects of PEEP and V_{T} on clearance depend on how these ventilation parameters combine to affect peak deflation rate and P_{AW.MAX}. With passive deflation, only ventilation with PEEP ≧5 cmH₂O and V_{T} of 12 ml/kg caused peak deflation rate to exceed 33 cmH₂O/sec (see FIG. 12B). With these combinations of PEEP and V_{T}, P_{AW.MAX} was high and clearance moderate (see FIG. 13D). With passive deflation and either PEEP of 15 cmH₂O and V_{T} of 6 ml/kg or PEEP of 0 cmH₂O and V_{T} of 12 ml/kg, peak deflation rate was 25-30 cmH₂O/sec. In the first of these cases, a high P_{AW.MAX} of 20 cmH₂O prevented clearance. But in the second case, a moderate P_{AW.MAX} of 11 cmH₂O enabled moderate clearance. With accelerated deflation, ventilation with PEEP of 0 cmH₂O and V_{T} of 6 ml/kg had a low peak deflation rate. With an also low P_{AW.MAX}, moderate clearance was achieved. Under all other ventilation conditions with accelerated deflation, peak deflation rate was >33 cmH₂O/sec. Amongst these groups, low PEEP minimized P_{AW.MAX} and maximized clearance. For these same groups, peak deflation generally occurred within one sampling period, 0.04 sec, of the start of deflation.

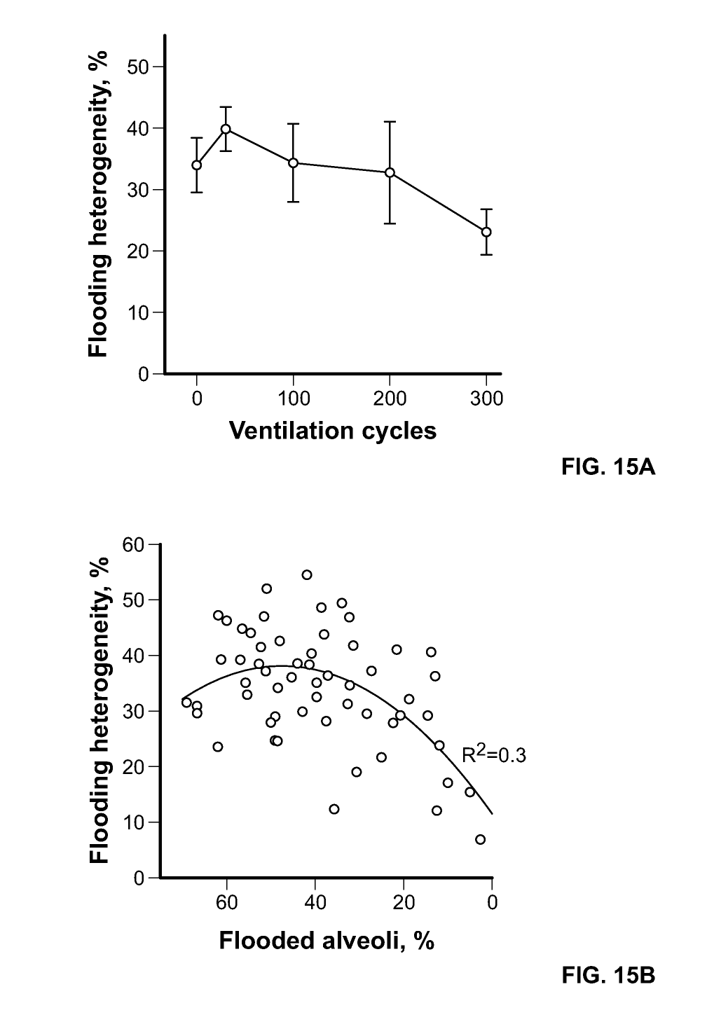

Finally, it was found that clearance tended initially to increase and then to decrease flooding heterogeneity. Heterogeneity was quantified over time in the three groups with greatest clearance and found to exhibit the same trend in each. This trend is shown for the group with PEEP of 5 cmH₂O, V_{T} of 6 ml/kg and accelerated deflation in FIG. 15A. The net decrease in heterogeneity was not significant after 300 cycles, but might become so at a later time point. A plot of flooding heterogeneity vs. % flooded alveoli that was generated with final-time point data from all groups shows that, overall, heterogeneity decreased as % flooding decreases (FIG. 15B). The plot also suggests a maximum in the heterogeneity-flooding relation—a quadric fit peaks at 47% flooding, close to the 50% flooding at which one would expect maximum heterogeneity. Clearance that reduces flooding below 50% should reduce heterogeneity, stress concentrations and ventilation injury.

I claim:

1. A method for reducing ventilator-induced injury, during mechanical ventilation, to a lung having heterogeneous alveolar flooding by promoting equitable redistribution of liquid amongst alveoli comprising effecting abrupt accelerated deflation of the lung while maintaining a zero end-expiratory pressure (ZEEP) or a positive end-expiratory pressure (PEEP).

2. The method of claim 1, further comprising applying vacuum pressure at an exit of an outflow path of a ventilator.

3. The method of claim 1, wherein a ZEEP is maintained.

4. The method of claim 3, wherein a PEEP is maintained and the PEEP is from greater than zero to about 20 centimeters of water (cmH₂O).

5. The method of claim 4, wherein the PEEP is from greater than zero to about 15 cmH₂O.

6. The method of claim 1, wherein the mechanical ventilation includes inflating the lung before causing accelerated deflation and wherein inflating the lung is accomplished using a tidal volume of about 12 milliliters per kilogram body weight (ml/kg) or less.

7. The method of claim 6, wherein inflating the lung is accomplished using a tidal volume of about 6 ml/kg or less.

8. A method for promoting equitable distribution of liquid amongst pulmonary alveoli in the presence of alveolar flooding with a ventilation means including a main conduit for fluidly connecting a source of ventilation gas to a lung, the main conduit having a receiving inlet for receiving ventilation gas from a source of ventilation gas and a discharge outlet for discharging ventilation gas to the lung, a deflation branch for fluidly connecting the main conduit to a source of atmospheric or vacuum pressure, an inflation proportional valve located along the main conduit between the receiving inlet and the deflation branch, a deflation proportional valve located along the deflation branch, and a pressure transducer for indicating the pressure within the main conduit, the pressure transducer being located along the main conduit between the deflation branch and the discharge outlet of the main conduit, said method including the steps of:
fluidly connecting the receiving inlet of the main conduit to a source of ventilation gas at a positive pressure, the deflation branch outlet to a source of atmospheric or vacuum pressure, and the discharge outlet of the main conduit to a lung having alveolar flooding;
inflating the lung to a target maximal pressure or with a target tidal volume with the ventilation gas;
closing the inflation proportional valve;
suddenly opening the deflation proportional valve, thereby effecting an abrupt accelerated deflation of the lung; and
holding the deflation proportional valve fully or partially open until the pressure transducer indicates a pressure in the main conduit that is equal to zero or a target PEEP, then closing the deflation proportional valve, thereby maintaining zero or positive pressure in the main conduit and the lung.

9. The method of claim 8, further comprising gradually opening the inflation proportional valve, thereby increasing the pressure in the main conduit above the target zero or positive end-expiratory pressure.

10. The method of claim 8, further comprising applying vacuum pressure at the deflation branch outlet during the suddenly opening step.

11. The method of claim 8, wherein the step of inflating the lung to target maximal pressure or with target tidal volume with the ventilation gas is performing using a tidal volume of ventilation gas of about 12 ml/kg or less.

12. The method of claim 11, wherein the step of inflating the lung to target maximal pressure or with target tidal volume with the ventilation gas is performing using a tidal volume of ventilation gas of about 6 ml/kg or less.

13. The method of claim 8, wherein a zero end-expiratory pressure is maintained.

14. The method of claim 8, wherein the target PEEP is from greater than zero to about 20 cmH₂O.

15. The method of claim 14, wherein the target PEEP is from greater than zero to about 15 cmH₂O.

16. An apparatus for promoting equitable distribution of liquid amongst pulmonary alveoli in the presence of alveolar flooding, comprising:
a main conduit for fluidly connecting a source of ventilation gas to a lung, the main conduit having a receiving inlet for receiving ventilation gas from a source of ventilation gas and a discharge outlet for discharging ventilation gas to the lung;
a deflation branch for fluidly connecting the main conduit to a source of atmospheric or vacuum pressure;
an inflation proportional valve, located along the main conduit between the receiving inlet of the main conduit and the deflation branch;
a deflation proportional valve, located along the deflation branch; and
a pressure transducer for indicating the pressure within the main conduit, the pressure transducer being located along the main conduit between the deflation branch and the discharge outlet of the main conduit.

17. The apparatus of claim 16 further comprising a software program, a digital/analog conversion device and proportional drivers wherein the software program acquires pressure data from the pressure transducer and provides voltage signals that control the proportional valves via the digital/analog conversion device and proportional drivers.
